(19) United States
(12) Patent Application Publication
(10) Pub. No.: US 2023/0220484 A1
(43) Pub. Date: Jul. 13, 2023

(54) Methods, Systems, and Compositions for the Analysis of Cell-Free Nucleic Acids

(71) Applicant: Sequenom, Inc., San Diego, CA (US)

(72) Inventors: Kimberly Holden, San Diego, CA (US); Taylor Jensen, San Diego, CA (US)

(21) Appl. No.: 17/925,297

(22) PCT Filed: May 14, 2021
(86) PCT No.: PCT/US2021/032526

§ 371 Date: Nov. 14, 2022

Related U.S. Application Data
(60) Provisional application No. 63/024,673, filed on May 14, 2020.

(51) Int. Cl. C12Q 1/6886

(52) U.S. Cl. CPC C12Q 1/6886; C12Q 2600/156; C12Q 1/6806

(57) ABSTRACT

The present disclosure relates to methods for enriching circulating tumor DNA (ctDNA) to enhance early disease detection or predictions of disease progression. The present disclosure also relates to methods for enriching circulating fetal cell free DNA (fetal cfDNA) to enhance early disease detection. In some embodiments, the method comprises enriching ctDNA or fetal cfDNA in a sample by selecting for cell-free nucleic acid fragments that are less than 150 bp prior to copy number alteration (CNA) analysis. Also disclosed are compositions, systems, and computer-program products for analyzing circulating cell free nucleic acids by any of the methods disclosed herein.

### RELATED APPLICATIONS

This application claims priority to U.S. Provisional Application No. 63/024,673, filed on May 14, 2020. The entire content of said provisional application is herein incorporated by reference for all purposes.

### FIELD OF INVENTION

This application is directed to methods, systems, and compositions for analyzing cell-free nucleic acids.

### BACKGROUND

Cell-free DNA (cfDNA) derived from tumor cells is present in the plasma of patients with cancer, and enriching for this circulating tumor DNA (ctDNA) can be useful in early disease detection or for predicting disease progression. However, the proportion of ctDNA is typically less than 2%. To overcome this limitation, current methodologies have sought to better distinguish the biological signal derived from ctDNA from the typically present technical and statistical noise. Unfortunately, these methods often require increased sequencing depth and other advanced analytical techniques. Thus, alternative or complimentary approaches would be beneficial for improving noninvasive cancer diagnostics (i.e. liquid biopsies). Additionally, these new approaches would be beneficial for improving non-invasive prenatal testing based on circulating fetal cell free DNA (fetal cfDNA).

### SUMMARY

Disclosed are methods, systems, computer-program products, and compositions for enriching circulating tumor DNA (ctDNA) to enhance early disease detection or predictions of disease progression. The present disclosure also relates to methods, systems, computer-program products, and compositions for enriching circulating fetal cell free DNA (fetal cfDNA) to enhance early disease detection. The methods, systems, computer-program products, and compositions may be embodied in a variety of ways.

In one embodiment, disclosed is a method for analyzing circulating cell-free nucleic acids from a subject comprising obtaining a sample comprising circulating cell-free nucleic acid fragments from the subject and preparing a library from the sample, wherein the library comprises the circulating cell-free nucleic acid fragments ligated to at least one adapter. The method may further comprise selecting for adapter-ligated nucleic acids having a subject cell-free nucleic acid fragment that is less than 150 bp. In some embodiments, the subject cell free nucleic acid fragments may be less than 143 bp. In some embodiments, the subject cell-free nucleic acid fragment may be greater than 15 bp.

The method may further comprise determining the sequence of the selected subject nucleic acid fragments. Additionally, the method may further comprise quantifying copy number alterations (CNAs) in the sequenced subject nucleic acid fragments.

In an embodiment of the method, the sample is a plasma sample. In some embodiments, the circulating cell-free nucleic acid fragments comprise circulating tumor DNA (ctDNA). In some embodiments, the circulating cell-free nucleic acid fragments comprise circulating fetal cell-free DNA (fetal cfDNA).

In some embodiments, the method may further comprise determining the status of the subject based on the CNAs present in the selected subject nucleic acid fragments. In an embodiment, the status of the subject can be a presence or absence of a cancer. In another embodiment, the status of the subject can be a progression of a cancer. In another embodiment, the status of the subject can be a remission of a cancer. In another embodiment, the status of the subject can be pregnant with a fetus exhibiting an aneuploidy.

In some embodiments, the level of the CNAs may be quantified using a genomic instability number (GIN).

In some embodiments, the adapter-ligated nucleic acid fragments can be size selected via electrophoresis. In some embodiments, the adapter-ligated nucleic acid fragments may be size selected via magnetic bead-based selection. In some embodiments, the adapter-ligated nucleic acid fragments may be size selected in silico during the processing of sequencing data.

In additional embodiments, disclosed are systems and computer program products for analyzing circulating cell free nucleic acids by any of the methods disclosed herein.

In additional embodiments, disclosed are compositions for analyzing circulating cell-free nucleic acids from a subject comprising a library of circulating cell-free nucleic acids ligated to at least one adaptor.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 shows a flow chart illustrating an embodiment of the disclosed methods.

FIG. 2 shows an illustrative embodiment of DNA fragments in a sample from a cancer patient wherein the fraction of ctDNA in the sample increase after size selection in accordance with an embodiment of the disclosure.

FIG. 3 shows the median cfDNA fragment size in a set of libraries before and after size selection in healthy subjects, subjects with cancer, pregnant subjects with a known euploid fetus, and pregnant subjects with a known trisomy 21 fetus in accordance with an embodiment of the disclosure. Results are reported using standard box and whisker plots showing the median, the boxes extending to the bounds of the lower and upper quartiles, and the lines indicating the variability outside of the upper and lower quartiles.

FIG. 4 shows the area under the curve (AUC) difference between the amplitudes of detectable autosomal copy number alterations (CNAs) before and after size selection in healthy subjects, subjects with cancer, pregnant subjects with a known euploid fetus, and pregnant subjects with a known trisomy 21 fetus in accordance with an embodiment of the disclosure. Results are reported using standard box and whisker plots.

FIG. 5 shows the average AUC difference of all detected CNAs in each of 16 cancer patients as the size selection cutoff is increased in accordance with an embodiment of the disclosure.

FIGS. 6A, 6B, and 6C, show an example enrichment of CNAs in a sample from a cancer patient following size selection using a 152 bp cutoff in accordance with an embodiment of the disclosure. FIG. 6A shows the genome-wide profiles of the sample before (top panel) and after (lower panel) size selection where CNAs increased in magnitude only slightly and the GIN increased some after size selection. FIG. 6B shows the cfDNA fragment size profile of the sample before and after size selection. FIG. 6C shows the absolute value of the AUC for each CNA detected pre-size selection on the left and post-size selection on the right.

FIGS. 7A, 7B, and 7C show an example enrichment of CNAs in a sample from a cancer patient following size selection using a 116 bp cutoff in accordance with an embodiment of the disclosure. FIG. 7A shows the genome-wide profiles of the sample before (upper panel) and after (lower panel) size selection where CNAs increased significantly in magnitude and the GIN increased significantly after size selection. FIG. 7B shows the cfDNA fragment size profile of the sample before and after size selection. FIG. 7C, shows copy number alterations post-size selection were amplified.

FIGS. 8A, 8B, and 8C show an example of a sample where the CNAs are likely germline as the AUC does not change much pre- and post-size selection in accordance with an embodiment of the disclosure. FIG. 8A shows the genome-wide profiles of the sample before (upper panel) and after (lower panel) size selection where CNAs did not change significantly in magnitude and the GIN did not change significantly after size selection. FIG. 8B shows the cfDNA fragment size profile of the sample before and after size selection. FIG. 8C, shows copy number alterations post-size selection not significantly different.

FIG. 9 shows an illustrative embodiment of a system in which certain embodiments of the technology may be implemented.

### DETAILED DESCRIPTION

The following description recites various aspects and embodiments of the present compositions and methods. No particular embodiment is intended to define the scope of the compositions and methods. Rather, the embodiments merely provide non-limiting examples of various methods and systems that are at least included within the scope of the compositions and methods. The description is to be read from the perspective of one of ordinary skill in the art; therefore, information well known to the skilled artisan is not necessarily included.

### Definitions

The present disclosure now will be described more fully hereinafter. The disclosure may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the present disclosure or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Methods

The present disclosure relates to methods for enriching circulating tumor DNA (ctDNA) to enhance early disease detection or predictions of disease progression. The present disclosure also relates to methods for enriching circulating fetal cell-free DNA (fetal cfDNA) to enhance early disease detection. The methods and systems may be embodied in a variety of ways.

In one embodiment, disclosed is a method for analyzing circulating cell-free nucleic acids from a subject comprising obtaining a sample comprising circulating cell-free nucleic acid fragments from the subject, preparing a library from the sample. In an embodiment, the library comprises the circulating cell-free nucleic acid fragments ligated to at least one adapter. The method may further comprise selecting for adapter-ligated nucleic acids having a subject cell-free nucleic acid fragment that is less than 150 bp. In some embodiments, the subject cell-free nucleic acid fragment may be greater than 15 bp. The method may further comprise determining the sequence of the selected subject nucleic acid fragments. Additionally, the method may further comprise quantifying copy number alterations (CNAs) in the sequenced subject nucleic acid fragments.

In some embodiments, the sample is a plasma sample. Or other sample types as disclosed herein may be used.

In some embodiments, the circulating cell-free nucleic acid fragments comprise ctDNA. In some embodiments, the circulating cell-free nucleic acid fragments comprise circulating fetal cfDNA. Or, other types of cell-free nucleic acid fragments may be used.

In some embodiments, the method further comprises determining the status of the subject based on the CNAs present in the selected subject nucleic acid fragments. For example, in some embodiments, the status of the subject is a presence or absence of a cancer. In other embodiments, the status of the subject is a progression of a cancer. In yet other embodiments, the status of the subject is a remission of a cancer. In other embodiments, the status of the subject is pregnant with a fetus exhibiting an aneuploidy.

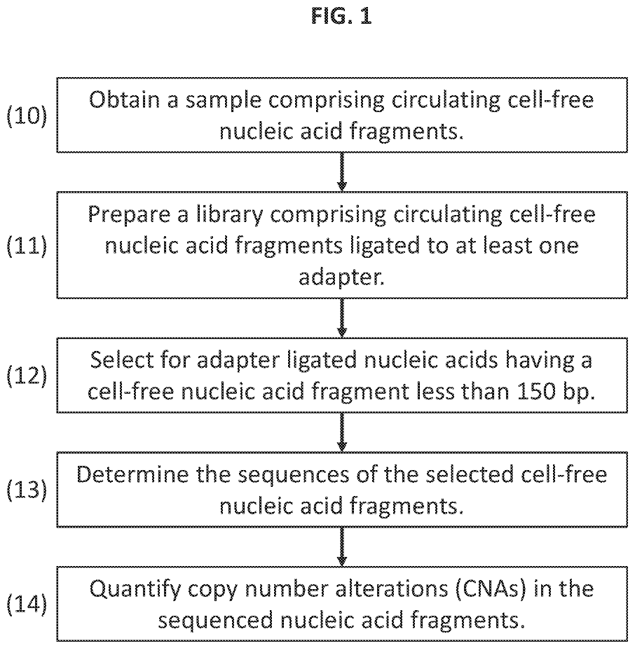

An embodiment of the method is illustrated in FIG. 1. Thus, the method may include the step (10) of obtaining a sample comprising circulating cell-free nucleic acid fragments from the subject. The method may further include the step (11) of preparing a library comprising the circulating cell-free nucleic acid fragments optionally ligated to at least one adapter. The method may further include the step (12) of selecting for adapter ligated nucleic acids having a cell-free nucleic acid fragment less than 150 bp. The method may also include the step (13) of determining the sequences of the selected cell-free nucleic acid fragments. Additionally, the method may include the step (14) of quantifying copy number alterations (CNAs) in the sequenced nucleic acid fragments.

Samples

Provided herein are methods, compositions, and systems for analyzing nucleic acids. In some embodiments, nucleic acid fragments in a mixture of nucleic acid fragments are analyzed. Nucleic acid fragments may be referred to as nucleic acid templates, and the terms may be used interchangeably herein. A mixture of nucleic acids can comprise two or more nucleic acid fragment species having the same or different nucleotide sequences, different fragment lengths, different origins (e.g., genomic origins, fetal vs. maternal origins, cell or tissue origins, cancer vs. non-cancer origin, tumor vs. non-tumor origin, sample origins, subject origins, and the like), or combinations thereof.

In some embodiments of the disclosed methods, compositions, and systems, the nucleic acid in a sample is from a subject. In some embodiments, the nucleic acid in a sample comprises circulating cell free nucleic acid. In some embodiments, circulating cell free nucleic acid is from blood plasma or blood serum from a test subject. Or, other biological samples as detailed herein may be used.

In some embodiments, a subject is a cancer patient, or is a subject being tested or screened for cancer. In some embodiments, nucleic acid in a sample comprises patient nucleic acid and tumor nucleic acid or nucleic acid from a cancer cell. In some embodiments, the fraction of tumor/cancer nucleic acid in a sample is less than about 25%. For example, the fraction of tumor/cancer nucleic acid in a sample may be about 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%. In some embodiments, the fraction of tumor/cancer nucleic acid in a sample is less than about 10%. In some embodiments, the fraction of tumor/cancer nucleic acid in a sample is less than about 5%.

In some embodiments, a subject is a pregnant female. In some embodiments, nucleic acid in a sample comprises maternal nucleic acid and fetal nucleic acid. In some embodiments, the fraction of fetal nucleic acid in a sample is less than about 25%. For example, the fraction of fetal nucleic acid in a sample may be about 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%. In some embodiments, the fraction of fetal nucleic acid in a sample is less than about 10%. In some embodiments, the fraction of fetal nucleic acid in a sample is less than about 5%.

Nucleic acid or a nucleic acid mixture utilized in the methods, compositions, and systems described herein often is isolated from a sample obtained from a subject (e.g., a test subject). A subject can be any living or non-living organism, including but not limited to a human, a non-human animal, a plant, a bacterium, a fungus, a protest or a pathogen. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman, a pregnant woman). A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen. In some embodiments, a test subject is a female. In some embodiments, a test subject is a human female. In some embodiments, a test subject is a male. In some embodiments, a test subject is a human male.

A sample can be a liquid sample. A liquid sample can comprise extracellular nucleic acid (e.g., circulating cell-free DNA). Non-limiting examples of liquid samples, include, blood or a blood product (e.g., serum, plasma, or the like), urine, a biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments of the disclosed methods, compositions, and systems, a biological sample may be blood, plasma, or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of fetal DNA found in maternal blood, for example, may be performed using, e.g., whole blood, serum, or plasma. An analysis of tumor DNA found in a patient's blood, for example, may be performed using, e.g., whole blood, serum, or plasma. Methods for preparing serum or plasma from blood obtained from a subject (e.g., a maternal subject; cancer patient) are known. For example, a subject's blood (e.g., a pregnant woman's blood; cancer patient's blood) can be placed in a tube containing EDTA, acid citrate dextrose (ACD), or a specialized commercial product such as Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

Nucleic Acid

Provided herein are methods, compositions, and systems for analyzing nucleic acid. The terms “nucleic acid,” “nucleic acid molecule,” “nucleic acid fragment,” and “nucleic acid template” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

Nucleic acid may be single or double stranded. Single stranded DNA, for example, can be generated by denaturing double stranded DNA by heating or by treatment with alkali, for example. In certain embodiments, nucleic acid is in a D-loop structure, formed by strand invasion of a duplex DNA molecule by an oligonucleotide or a DNA-like molecule such as peptide nucleic acid (PNA). D loop formation can be facilitated by addition of E. Coli RecA protein and/or by alteration of salt concentration, for example, using methods known in the art.

Nucleic acid provided for the methods, compositions, and systems described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

Nucleic acid may be isolated from a sample by methods known in the art. Any suitable method can be used for isolating, extracting and/or purifying DNA from a biological sample (e.g., from blood or a blood product), non-limiting examples of which include methods of DNA preparation (e.g., described by Sambrook and Russell, Molecular Cloning: A Laboratory Manual 3d ed., 2001), various commercially available reagents or kits, such as Qiagen's QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), and GFX™ Genomic Blood DNA Purification Kit (Amersham, Piscataway, N.J.), the like or combinations thereof.

Nucleic acids can include extracellular nucleic acid in certain embodiments. The term “extracellular nucleic acid” as used herein can refer to nucleic acid isolated from a source having substantially no cells and also is referred to as “cell-free” nucleic acid, “circulating cell-free nucleic acid” (e.g., CCF fragments, ccf DNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. As used herein, the term “obtain cell-free circulating sample nucleic acid” includes obtaining a sample directly (e.g., collecting a sample, e.g., a test sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”). In some embodiments, sample nucleic acid from a subject is circulating cell-free nucleic acid. In some embodiments, circulating cell free nucleic acid is from blood plasma or blood serum from a subject.

Extracellular nucleic acid can include different nucleic acid species, and therefore is referred to herein as “heterogeneous” in certain embodiments. For example, blood serum or plasma from a person having cancer can include nucleic acid from cancer cells (e.g., tumor, neoplasia) and nucleic acid from non-cancer cells. In another example, blood serum or plasma from a pregnant female can include maternal nucleic acid and fetal nucleic acid. In some instances, cancer or fetal nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or 51% of the total nucleic acid is cancer or fetal nucleic acid).

At least two different nucleic acid species can exist in different amounts in extracellular nucleic acid and sometimes are referred to as minority species and majority species. In certain instances, a minority species of nucleic acid is from an affected cell type (e.g., cancer cell, wasting cell, cell attacked by immune system) and a majority species is from a normal (i.e., healthy cell). In certain instances, a minority species of nucleic acid is from a fetal cell and a majority species is from a maternal cell. In certain embodiments, a genetic variation or genetic alteration (e.g., copy number alteration, copy number variation, single nucleotide alteration, single nucleotide variation, chromosome alteration, and/or translocation) is determined for a minority nucleic acid species. In certain embodiments, a genetic variation or genetic alteration is determined for a majority nucleic acid species. Generally it is not intended that the terms “minority” or “majority” be rigidly defined in any respect. In one aspect, a nucleic acid that is considered “minority,” for example, can have an abundance of at least about 0.1% of the total nucleic acid in a sample to less than 50% of the total nucleic acid in a sample. In some embodiments, a minority nucleic acid can have an abundance of at least about 1% of the total nucleic acid in a sample to about 40% of the total nucleic acid in a sample. In some embodiments, a minority nucleic acid can have an abundance of at least about 2% of the total nucleic acid in a sample to about 30% of the total nucleic acid in a sample. In some embodiments, a minority nucleic acid can have an abundance of at least about 3% of the total nucleic acid in a sample to about 25% of the total nucleic acid in a sample. For example, a minority nucleic acid can have an abundance of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29% or 30% of the total nucleic acid in a sample. In some instances, a minority species of extracellular nucleic acid sometimes is about 1% to about 40% of the overall nucleic acid (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% of the nucleic acid is minority species nucleic acid). In some embodiments, the minority nucleic acid is extracellular DNA. In some embodiments, the minority nucleic acid is extracellular DNA from apoptotic tissue. In some embodiments, the minority nucleic acid is extracellular DNA from tissue affected by a cell proliferative disorder. In some embodiments, the minority nucleic acid is extracellular DNA from a tumor cell. In some embodiments, the minority nucleic acid is extracellular fetal DNA.

In another aspect, a nucleic acid that is considered “majority,” for example, can have an abundance greater than 50% of the total nucleic acid in a sample to about 99.9% of the total nucleic acid in a sample. In some embodiments, a majority nucleic acid can have an abundance of at least about 60% of the total nucleic acid in a sample to about 99% of the total nucleic acid in a sample. In some embodiments, a majority nucleic acid can have an abundance of at least about 70% of the total nucleic acid in a sample to about 98% of the total nucleic acid in a sample. In some embodiments, a majority nucleic acid can have an abundance of at least about 75% of the total nucleic acid in a sample to about 97% of the total nucleic acid in a sample. For example, a majority nucleic acid can have an abundance of at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the total nucleic acid in a sample. In some embodiments, the majority nucleic acid is extracellular DNA. In some embodiments, the majority nucleic acid is extracellular maternal DNA. In some embodiments, the majority nucleic acid is DNA from healthy tissue. In some embodiments, the majority nucleic acid is DNA from non-tumor cells.

In some embodiments, a minority species of extracellular nucleic acid is of a length of about 200 base pairs or less (e.g., about 70, 75, 80, 85, 90, 91, 92, 93, 94, 95, 96, 97, 98, 99 or 100% of minority species nucleic acid is of a length of about 200 base pairs or less). In some embodiments, a minority species of extracellular nucleic acid is of a length of about 150 base pairs or less (e.g., about 70, 75, 80, 85, 90, 91, 92, 93, 94, 95, 96, 97, 98, 99 or 100% of minority species nucleic acid is of a length of about 150 base pairs or less). In some embodiments, a minority species of extracellular nucleic acid is of a length of about 143 base pairs or less (e.g., about 70, 75, 80, 85, 90, 91, 92, 93, 94, 95, 96, 97, 98, 99 or 100% of minority species nucleic acid is of a length of about 143 base pairs or less). In some embodiments, a minority species of extracellular nucleic acid is of a length of about 100 base pairs or less (e.g., about 70, 75, 80, 85, 90, 91, 92, 93, 94, 95, 96, 97, 98, 99 or 100% of minority species nucleic acid is of a length of about 100 base pairs or less). In some embodiments, a minority species of extracellular nucleic acid is of a length of about 50 base pairs or less (e.g., about 70, 75, 80, 85, 90, 91, 92, 93, 94, 95, 96, 97, 98, 99 or 100% of minority species nucleic acid is of a length of about 50 base pairs or less).

In some embodiments, a minority species of extracellular nucleic acid is of a length of at least 10 base pairs or more (e.g., about 70, 75, 80, 85, 90, 91, 92, 93, 94, 95, 96, 97, 98, 99 or 100% of minority species nucleic acid is of a length of about 10 base pairs or more. In some embodiments, a minority species of extracellular nucleic acid is of a length at least 15 base pairs or more (e.g., about 70, 75, 80, 85, 90, 91, 92, 93, 94, 95, 96, 97, 98, 99 or 100% of minority species nucleic acid is of a length of about 15 base pairs or more). In some embodiments, a minority species of extracellular nucleic acid is of a length of about 20 base pairs or more (e.g., about 70, 75, 80, 85, 90, 91, 92, 93, 94, 95, 96, 97, 98, 99 or 100% of minority species nucleic acid is of a length of about 20 base pairs or more).

Enriching Nucleic Acids

In some embodiments of the disclosed methods, compositions, and systems, nucleic acid (e.g., extracellular nucleic acid) is enriched or relatively enriched for a subpopulation or species of nucleic acid. Nucleic acid subpopulations can include, for example, fetal nucleic acid, maternal nucleic acid, cancer nucleic acid, patient nucleic acid, minority nucleic acid, nucleic acid comprising fragments of a particular length or range of lengths, or nucleic acid from a particular genome region (e.g., single chromosome, set of chromosomes, and/or certain chromosome regions). Such enriched samples can be used in conjunction with a method provided herein. Thus, in certain embodiments, methods, compositions, and systems of the technology comprise enriching for a subpopulation of nucleic acid in a sample, such as, for example, cancer or fetal nucleic acid or other minority nucleic acids. In certain embodiments, a method for determining fraction of cancer cell nucleic acid or fetal fraction also can be used to enrich for cancer or fetal nucleic acid. In certain embodiments, nucleic acid from normal tissue (e.g., non-cancer cells) is selectively removed (partially, substantially, almost completely or completely) from the sample. In certain embodiments, maternal nucleic acid is selectively removed (partially, substantially, almost completely or completely) from the sample. In certain embodiments, enriching for a particular low copy number species nucleic acid (e.g., cancer or fetal nucleic acid) may improve quantitative sensitivity.

In certain embodiments of the disclosed methods, compositions, and systems, nucleic acid is enriched for a specific nucleic acid fragment length or range of fragment lengths using one or more length-based separation methods described below. In some embodiments, the adapter-ligated nucleic acid fragments are size selected in vitro via electrophoresis. In some embodiments, the adapter-ligated nucleic acid fragments are size selected via magnetic bead-based selection. In some embodiments, the adapter-ligated nucleic acid fragments are size selected in silico during the processing of sequencing data. In other embodiments, nucleic acid is enriched for fragments from a select genomic region (e.g., chromosome) using one or more sequence-based separation methods described herein and/or known in the art.

In some embodiments, nucleic acid is enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also is sometimes referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). See, e.g., Mouliere et al., Enhanced detection of circulating tumor DNA by fragment size analysis, 10 Sci. Transl. Med., eeat4921 (2018); see also U.S. Pat. Nos. 9,738,931; 7,838,647; 9,580,751. In certain instances, length-based separation approaches can include selective sequence tagging approaches, fragment circularization, chemical treatment (e.g., formaldehyde, polyethylene glycol (PEG) precipitation), mass spectrometry and/or size-specific nucleic acid amplification, for example.

Nucleic Acid Library

In some embodiments, the disclosed methods, compositions, and systems comprise a nucleic acid library. In some embodiments, a nucleic acid library is a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process. A nucleic acid library can comprise a nucleic acid derived from a single sample or multiplexed samples.

In some embodiments, a library of nucleic acids is modified to comprise a chemical moiety (e.g., a functional group) configured for immobilization of nucleic acids to a solid support. In some embodiments a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component C1q, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, the like or combinations thereof. Polynucleotides of known sequence can be added at a suitable position, for example on the 5′ end, 3′ end or within a nucleic acid sequence. Polynucleotides of known sequence can be the same or different sequences. In some embodiments a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments a library of nucleic acid can comprise chromosome-specific tags, capture sequences, labels and/or adapters. In some embodiments, a library of nucleic acids comprises one or more detectable labels. In some embodiments one or more detectable labels may be incorporated into a nucleic acid library at a 5′ end, at a 3′ end, and/or at any nucleotide position within a nucleic acid in the library. In some embodiments a library of nucleic acids comprises hybridized oligonucleotides. In certain embodiments hybridized oligonucleotides are labeled probes. In some embodiments a library of nucleic acids comprises hybridized oligonucleotide probes prior to immobilization on a solid phase.

In some embodiments, a polynucleotide of known sequence comprises a universal sequence. A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments two (e.g., a pair) or more universal sequences and/or universal primers are used. A universal primer often comprises a universal sequence. In some embodiments adapters (e.g., universal adapters) comprise universal sequences. In some embodiments one or more universal sequences are used to capture, identify and/or detect multiple species or subsets of nucleic acids.

In certain embodiments of preparing a nucleic acid library, (e.g., in certain sequencing by synthesis procedures), nucleic acids are size selected and/or fragmented into lengths of several hundred base pairs, or less (e.g., in preparation for library generation). In some embodiments, library preparation is performed without fragmentation (e.g., when using cell-free DNA).

In certain embodiments, a ligation-based library preparation method is used (e.g., ILLUMINA TRUSEQ, Illumina, San Diego Calif.). Ligation-based library preparation methods often make use of an adapter (e.g., a methylated adapter) design which can incorporate an index sequence (e.g., a sample index sequence to identify sample origin for a nucleic acid sequence) at the initial ligation step and often can be used to prepare samples for single-read sequencing, paired-end sequencing and multiplexed sequencing. For example, nucleic acids (e.g., fragmented nucleic acids or cell-free DNA) may be end repaired by a fill-in reaction, an exonuclease reaction or a combination thereof. In some embodiments the resulting blunt-end repaired nucleic acid can then be extended by a single nucleotide, which is complementary to a single nucleotide overhang on the 3′ end of an adapter/primer. Any nucleotide can be used for the extension/overhang nucleotides.

In some embodiments, nucleic acid library preparation comprises ligating an adapter oligonucleotide (e.g., to a sample nucleic acid, to a sample nucleic acid fragment, to a template nucleic acid). Adapter oligonucleotides are often complementary to flow-cell anchors, and sometimes are utilized to immobilize a nucleic acid library to a solid support, such as the inside surface of a flow cell, for example. An adapter oligonucleotide may, in certain embodiments, comprise an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). In some embodiments, an adapter oligonucleotide comprises one or more of primer annealing polynucleotide (e.g., for annealing to flow cell attached oligonucleotides and/or to free amplification primers), an index polynucleotide (e.g., sample index sequence for tracking nucleic acid from different samples; also referred to as a sample ID), and a barcode polynucleotide (e.g., single molecule barcode (SMB) for tracking individual molecules of sample nucleic acid that are amplified prior to sequencing; also referred to as a molecular barcode). Additionally and/or alternatively, a primer annealing component of an adapter oligonucleotide may comprise one or more universal sequences (e.g., sequences complementary to one or more universal amplification primers). In some embodiments, an index polynucleotide (e.g., sample index; sample ID) is a component of an adapter oligonucleotide. In some embodiments, an index polynucleotide (e.g., sample index; sample ID) is a component of a universal amplification primer sequence.

In some embodiments of the disclosed methods, compositions, and systems, adapter oligonucleotides when used in combination with amplification primers (e.g., universal amplification primers) are designed to generate library constructs comprising one or more of: universal sequences, molecular barcodes, sample ID sequences, spacer sequences, and a sample nucleic acid sequence. In some embodiments, adapter oligonucleotides when used in combination with universal amplification primers are designed generate library constructs comprising an ordered combination of one or more of: universal sequences, molecular barcodes, sample ID sequences, spacer sequences, and a sample nucleic acid sequence. For example, a library construct may comprise a first universal sequence, followed by a second universal sequence, followed by first molecular barcode, followed by a spacer sequence, followed by a template sequence (e.g., sample nucleic acid sequence), followed by a spacer sequence, followed by a second molecular barcode, followed by a third universal sequence, followed by a sample ID, followed by a fourth universal sequence. In some embodiments, adapter oligonucleotides when used in combination with amplification primers (e.g., universal amplification primers) are designed to generate library constructs for each strand of a template molecule (e.g., sample nucleic acid molecule). In some embodiments, adapter oligonucleotides are duplex adapter oligonucleotides.

In certain embodiments of the methods, compositions and systems, the library may comprise identifier nucleic acids. An identifier can be a suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments an identifier (e.g., a nucleic acid index or barcode) is a unique, known and/or identifiable sequence of nucleotides or nucleotide analogues. In some embodiments identifiers are six or more contiguous nucleotides. A multitude of fluorophores are available with a variety of different excitation and emission spectra. Any suitable type and/or number of fluorophores can be used as an identifier. In some embodiments 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In some embodiments of the disclosed methods, compositions, and systems, a nucleic acid library or parts thereof are amplified (e.g., amplified by a polymerase chain reaction (i.e., PCR)-based method). In some embodiments a sequencing method comprises amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments a rolling circle amplification method is used. In some embodiments amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

Genome Instability Number

The disclosed methods, compositions, and systems may be utilize various analytical methods. For example, in some embodiments, the level of the CNAs are quantified using a genomic instability number (GIN). Methods for assessing GIN are described, for example, in U.S. patent application Ser. No. 15/661,942, the entire content of which is incorporated herein by reference, including all text, tables, equations and drawings. Or, other methods of analyzing nucleic acids may be used. For example, in some cases DNA sequencing may be used to identify the source (e.g., minority vs. majority nucleic acids).

Briefly, for assessing GIN, sequencing reads may be mapped to the human reference genome (e.g., hg19) and partitioned in to 50 kbp non-overlapping segments. Or, other sized segments may be used. Regions are selected, and data is normalized as previously performed for noninvasive detection of fetal copy-number variants, Dharajiya et al., Incidental detection of maternal neoplasia in noninvasive prenatal testing, 64 Clin. Chem. 329-35 (2018); Zhao et al., Detection of fetal subchromosomal abnormalities by sequencing circulating cell-free DNA from maternal plasma, 61 Clin. Chem. 608-16 (2015), and the resultant normalized values are used to calculate a genome instability number (GIN). The GIN is a metric intended to capture genome-wide autosomal deviation from empirically derived euploid dosage of the genome in circulation. The GIN is a nonnegative, continuous value calculated as the absolute deviation of observed normalized sequencing read coverage from expected normalized read coverage summed across a defined number (e.g., 50,034) autosomal segments. In certain embodiments, fewer or more segments may be used. Observed normalized read coverage is defined for each genomic segment by an autosome-specific LOESS fit of the normalized data. The data can be represented as:

$$GIN = {\sum }_{i = 1}^{50034} \vert {fit}_{i} - {exp}_{i} \vert$$

where the GIN is defined as the sum across all autosomal bins, i, of the absolute deviation of LOESS fit of the normalized genomic representation of a sample, fitᵢ, to the expected normalized genomic representation of a sample without CNAs present, expᵢ. Increasing values of GIN are indicative of increasing deviation relative to an expected normal genomic profile.

### Systems

In additional embodiments, disclosed are systems (e.g., software) for analyzing circulating cell free nucleic acids by any of the steps of the methods or for generating or using any of the compositions disclosed herein. Also disclosed is a computer-program product tangibly embodied in a non-transitory machine-readable storage medium, including instructions configured to run the systems and/or perform a step or steps of the methods, and/or generating or using any of the disclosed compositions. For example, disclosed is a system and/or computer-program product for analyzing circulating cell-free nucleic acids from a subject by obtaining a sample comprising circulating cell-free nucleic acid fragments from the subject and preparing a library from the sample. In certain embodiments, the library comprises the circulating cell-free nucleic acid fragments ligated to at least one adapter. In some embodiments, the system and/or computer-program product may select for adapter-ligated nucleic acids having a subject cell-free nucleic acid fragment that is less than 150 bp. In some embodiments, the system and/or computer-program product may select for adapter-ligated nucleic acids having a subject cell free nucleic acid fragments that may be less than 143 bp. In some embodiments, the system and/or computer-program product may select for adapter-ligated nucleic acids having a subject cell-free nucleic acid fragment that may be greater than 15 bp.

The system and/or computer-program product may also determine the sequence of the selected subject nucleic acid fragments. Additionally, the system and/or computer-program product may quantify copy number alterations (CNAs) in the sequenced subject nucleic acid fragments.

In an embodiment of the system and/or computer-program product, the sample is a plasma sample. In some embodiments, the circulating cell-free nucleic acid fragments comprise circulating tumor DNA (ctDNA). In some embodiments, the circulating cell-free nucleic acid fragments comprise circulating fetal cell-free DNA (fetal cfDNA). Or, other sample types as disclosed herein may be used.

In some embodiments, the system and/or computer-program product may also determine the status of the subject based on the CNAs present in the selected subject nucleic acid fragments. In an embodiment, the status of the subject can be a presence or absence of a cancer. In another embodiment, the status of the subject can be a progression of a cancer. In another embodiment, the status of the subject can be a remission of a cancer. In another embodiment, the status of the subject can be pregnant with a fetus exhibiting an aneuploidy.

In some embodiments, the system and/or computer-program product may quantify the level of the CNAs using a genomic instability number (GIN).

In some embodiments, the system and/or computer-program product size selects adapter-ligated nucleic acid fragments via electrophoresis. In some embodiments, the system and/or computer-program product size selects the adapter-ligated nucleic acid fragments via magnetic bead-based selection. In some embodiments, the system and/or computer-program product size selects the adapter-ligated nucleic acid fragments in silico during the processing of sequencing data.

### Computer Systems and Computer Program Products

Certain processes and methods described herein often cannot be performed without a computer, microprocessor, software, module or other machine. At least certain steps of methods described herein, or systems described herein, may be computer-implemented, and one or more portions of a method sometimes are performed by one or more processors (e.g., microprocessors), computers, systems, apparatuses, or machines (e.g., microprocessor-controlled machine). For example, any of the steps of obtaining cell-free nucleic acids, preparing a library, characterizing the library, size selecting nucleic acid fragments, sequence determination and/or analysis (e.g., CNA determination or other analysis) may be performed at least in part using the systems and/or computer program products disclosed herein.

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein is a computer system configured to perform the any of the embodiments of the methods, or particular steps of any of the methods for analyzing circulating cell-free nucleic acids or to generate any of the disclosed compositions. In some embodiments, this invention provides a system for analyzing a library of circulating cell-free nucleic acids comprising one or more processors and non-transitory machine readable storage medium and/or memory coupled to one or more processors, and the memory or the non-transitory machine readable storage medium encoded with a set of instructions configured to perform a process.

Also provided herein are computer readable storage media with an executable program stored thereon, where the program instructs a microprocessor to perform any of the methods or method steps, and/or developing compositions comprising a library cell-free nucleic acid fragments described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

In some embodiments, the invention provides a non-transitory machine readable storage medium comprising program instructions that when executed by one or more processors cause the one or more processors to perform any of the methods disclosed herein.

Thus, also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Sequence reads, counts, levels and/or measurements sometimes are referred to as “data” or “data sets.” In some embodiments, data or data sets can be characterized by one or more features or variables (e.g., sequence based (e.g., GC content, specific nucleotide sequence, the like), function specific (e.g., expressed genes, cancer genes, the like), location based (genome specific, chromosome specific, portion or portion-specific), the like and combinations thereof). In certain embodiments, data or data sets can be organized into a matrix having two or more dimensions based on one or more features or variables. Data organized into matrices can be organized using any suitable features or variables. In certain embodiments, data sets characterized by one or more features or variables sometimes are processed after counting.

Machines, software and interfaces may be used to conduct any steps of the methods and/or to generate any of the compositions described herein. Using machines, software and interfaces, a user may enter, request, query or determine options for using particular information, programs or processes, which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more machines and/or stations for performing certain steps of the disclosed methods or for generating the disclosed compositions. Each machine may comprise one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine a classification outcome from the sequence reads.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, cloud or web-based systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, simulated data is generated by an in silico process and the simulated data serves as data that can be input via an input device. The term “in silico” refers to research and experiments performed using a computer.

A system may include software useful for performing a process or part of a process described herein, and software can include one or more modules for performing such processes (e.g., sequencing module, logic processing module, and data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein.

A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A module can perform one or more of the following non-limiting functions: mapping sequence reads, providing counts, assembling portions, providing or determining a level, providing a count profile, normalizing (e.g., normalizing reads, normalizing counts, and the like), providing a normalized count profile or levels of normalized counts, comparing two or more levels, providing uncertainty values, providing or determining expected levels and expected ranges (e.g., expected level ranges, threshold ranges and threshold levels), providing adjustments to levels (e.g., adjusting a first level, adjusting a second level, and/or padding), providing a statistical assessment as for example, but not limited to, determining a GIN, providing identification (e.g., identifying a genetic variation/genetic alteration or CNA), categorizing, plotting, and/or determining an outcome, for example. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product may be embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining the presence or absence of a genetic variation or genetic alteration.

A machine, in some embodiments, comprises at least one microprocessor for carrying out the instructions in a module. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., ipads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another module.

Software comprising program instructions often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, FORTRAN, and the like. In some embodiments, an algorithm can be configured or modified to include margin of errors, statistical analysis, statistical significance, and/or comparison to other information or data sets (e.g., applicable when using, for example, algorithms to analyze a library of cell-free nucleic acid fragments, such as a fixed cutoff algorithm, a dynamic clustering algorithm, or an individual polymorphic nucleic acid target threshold algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity. An algorithm with the highest sensitivity and/or specificity may be identified and utilized.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

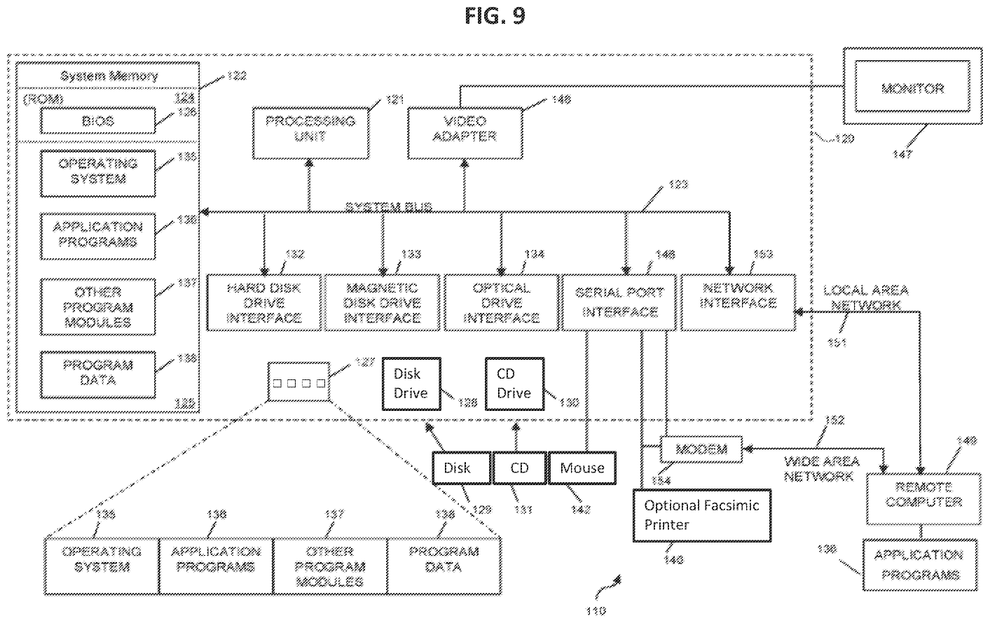

FIG. 9 illustrates a non-limiting example of a computing environment 110 in which various systems, methods, algorithms, and data structures described herein may be implemented. The computing environment 110 is only one example of a suitable computing environment and is not intended to suggest any limitation as to the scope of use or functionality of the systems, methods, and data structures described herein. Neither should computing environment 110 be interpreted as having any dependency or requirement relating to any one or combination of components illustrated in computing environment 110. A subset of systems, methods, and data structures shown in FIG. 9 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The operating environment 110 of FIG. 9 includes a general purpose computing device in the form of a computer 120, including a processing unit 121, a system memory 122, and a system bus 123 that operatively couples various system components including the system memory 122 to the processing unit 121. There may be only one or there may be more than one processing unit 121, such that the processor of computer 120 includes a single central-processing unit (CPU), or a plurality of processing units, commonly referred to as a parallel processing environment. The computer 120 may be a conventional computer, a distributed computer, or any other type of computer.

The system bus 123 may be any of several types of bus structures including a memory bus or memory controller, a peripheral bus, and a local bus using any of a variety of bus architectures. The system memory may also be referred to as simply the memory, and includes read only memory (ROM) 124 and random access memory (RAM). A basic input/output system (BIOS) 126, containing the basic routines that help to transfer information between elements within the computer 120, such as during start-up, is stored in ROM 124. The computer 120 may further include a hard disk drive interface 127 for reading from and writing to a hard disk, not shown, a magnetic disk drive 128 for reading from or writing to a removable magnetic disk 129, and an optical disk drive 130 for reading from or writing to a removable optical disk 131 such as a CD ROM or other optical media.

The hard disk drive 127, magnetic disk drive 128, and optical disk drive 130 may be connected to the system bus 123 by a hard disk drive interface 132, a magnetic disk drive interface 133, and an optical disk drive interface 134, respectively. The drives and their associated computer-readable media provide nonvolatile storage of computer-readable instructions, data structures, program modules and other data for the computer 120. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk, magnetic disk 129, optical disk 131, ROM 124, or RAM, including an operating system 135, one or more application programs 136, other program modules 137, and program data 138. A user may enter commands and information into the personal computer 120 through input devices such as a keyboard 140 and pointing device 142. Other input devices (not shown) may include a microphone, joystick, game pad, satellite dish, scanner, or the like. These and other input devices are often connected to the processing unit 121 through a serial port interface 146 that is coupled to the system bus, but may be connected by other interfaces, such as a parallel port, game port, or a universal serial bus (USB). A monitor 147 or other type of display device may be connected to the system bus 123 via an interface, such as a video adapter 148. In addition to the monitor, computers typically include other peripheral output devices (not shown), such as speakers and printers.

The computer 120 may operate in a networked environment using logical connections to one or more remote computers, such as remote computer 149. These logical connections may be achieved by a communication device coupled to or a part of the computer 120, or in other manners. The remote computer 149 may be another computer, a server, a router, a network PC, a client, a peer device or other common network node, and typically includes many or all of the elements described above relative to the computer 120, although only a memory storage device 150 has been illustrated in FIG. 9. The logical connections depicted in FIG. 9 include a local-area network (LAN) 151 and a wide-area network (WAN) 152. Such networking environments are commonplace in office networks, enterprise-wide computer networks, intranets and the Internet, which all are types of networks.

When used in a LAN-networking environment, the computer 120 is connected to the local network 151 through a network interface or adapter 153, which is one type of communications device. When used in a WAN-networking environment, the computer 120 often includes a modem 154, a type of communications device, or any other type of communications device for establishing communications over the wide area network 152. The modem 154, which may be internal or external, is connected to the system bus 123 via the serial port interface 146. In a networked environment, program modules depicted relative to the personal computer 120, or portions thereof, may be stored in the remote memory storage device. It is appreciated that the network connections shown are non-limiting examples and other communications devices for establishing a communications link between computers may be used.

### Compositions

Also disclosed herein are compositions. In certain embodiments, the compositions may be used for analyzing circulating cell-free nucleic acid from a subject. For example, in certain embodiments, disclosed are compositions for analyzing circulating cell-free nucleic acids from a subject comprising a library of circulating cell-free nucleic acids. In some embodiments, the composition comprises a library comprising adapter-ligated cell-free nucleic acid fragments that are less than 165 bp, or optionally less than 160 bp, or optionally less than 155 bp, or optionally less than 150 bp, or optionally less than 145 bp. In some embodiments, the composition comprises a library comprising adapter-ligated nucleic acids having a subject cell-free nucleic acid fragment that is greater than 15 bp

In some embodiments, the library further comprises adapter oligonucleotides ligated to a sample nucleic acid, to a sample nucleic acid fragment, or to a template nucleic acid. Adapter oligonucleotides are often complementary to flow-cell anchors, and sometimes are utilized to immobilize a nucleic acid library to a solid support, such as the inside surface of a flow cell, for example. An adapter oligonucleotide may, in certain embodiments, comprise an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). In some embodiments, an adapter oligonucleotide comprises one or more of primer annealing polynucleotide (e.g., for annealing to flow cell attached oligonucleotides and/or to free amplification primers), an index polynucleotide (e.g., sample index sequence for tracking nucleic acid from different samples; also referred to as a sample ID), and a barcode polynucleotide (e.g., single molecule barcode (SMB) for tracking individual molecules of sample nucleic acid that are amplified prior to sequencing; also referred to as a molecular barcode). Additionally and/or alternatively, a primer annealing component of an adapter oligonucleotide may comprise one or more universal sequences (e.g., sequences complementary to one or more universal amplification primers). In some embodiments, an index polynucleotide (e.g., sample index; sample ID) is a component of an adapter oligonucleotide. In some embodiments, an index polynucleotide (e.g., sample index; sample ID) is a component of a universal amplification primer sequence.

The following examples of specific embodiments of the invention are offered for illustrative purposes only, and are not intended to limit the scope of the invention in any way.

### EXAMPLES

### Example 1

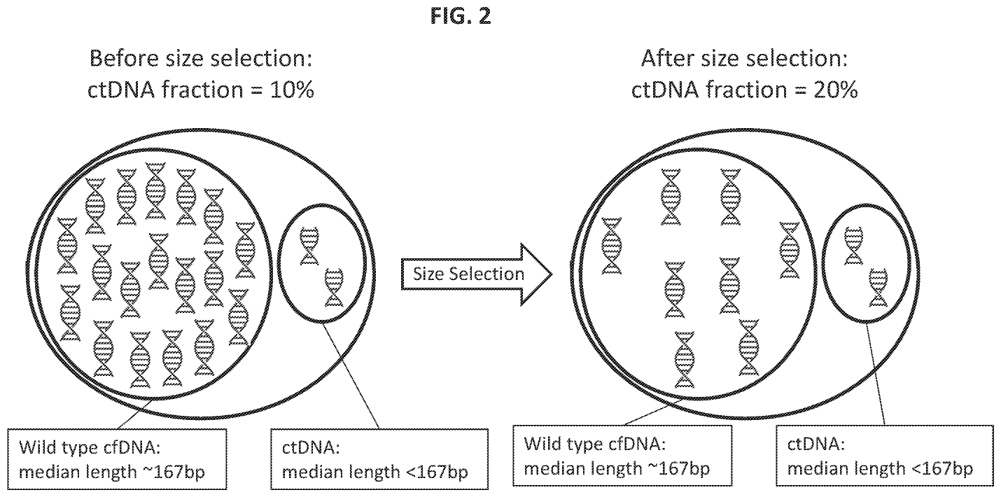

As is shown in FIG. 2, the DNA pool before size selection contains a large fraction of wild type cfDNA fragments, which, in this example, have a median length of about 167 bp. There are also a few ctDNA fragments, which have a median length less than 167 bp. In this example, the tumor fraction in this sample before size selection is 10%. Performing size selection results in a greater proportion of tumor fragments relative to wild type and increases the ctDNA fraction to 20%.

### Example 2

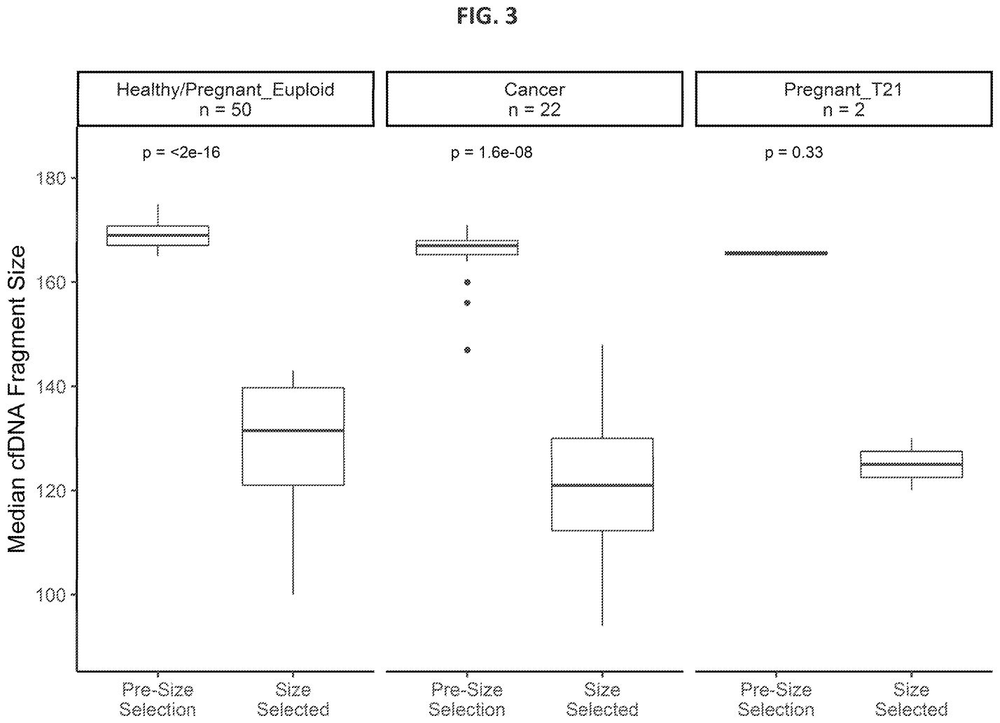

Cell free DNA (cfDNA) was size selected to determine if enriching shorter circulating tumor DNA (ctDNA) fragments enriched the signal for the detection of tumor-specific variants. The median length of cfDNA in circulation from healthy tissue is typically about 167 bp, while ctDNA has been demonstrated to be, on average, shorter. To test this hypothesis, adapter-ligated libraries were size selected using the Coastal Genomics NIMBUS Select, an automated platform for gel-based electrophoresis and size selection, targeting cfDNA fragment sizes up to 142 bp (+/−15 bp). The size selected libraries from each patient were first assayed with low-coverage (˜0.3×) genome-wide sequencing and analyzed for insert size to ensure proper enrichment of shorter cfDNA fragments. As is shown in FIG. 3, libraries prior to size selection had an average median cfDNA fragment size of 169 bp in samples from healthy patients and pregnant patients with known euploid fetuses, 165 bp in samples from cancer patients, and 165 bp in samples from pregnant patients with a fetus with a known trisomy on chromosome 21. After size selection the average median cfDNA fragment sizes were 129 bp in samples from healthy patients and pregnant patients with known euploid fetuses, 120 bp in samples from cancer patients, and 125 bp in samples from pregnant patients with a fetus with a known trisomy on chromosome 21. In all sample types, libraries prior to size selection yielded, on average, 24.5% of reads with cfDNA fragment sizes shorter than 150 bp. After size selection, the proportion of cfDNA shorter than 150 bp was significantly increased to 92.0% (p<0.001; Wilcoxon Rank Sum).

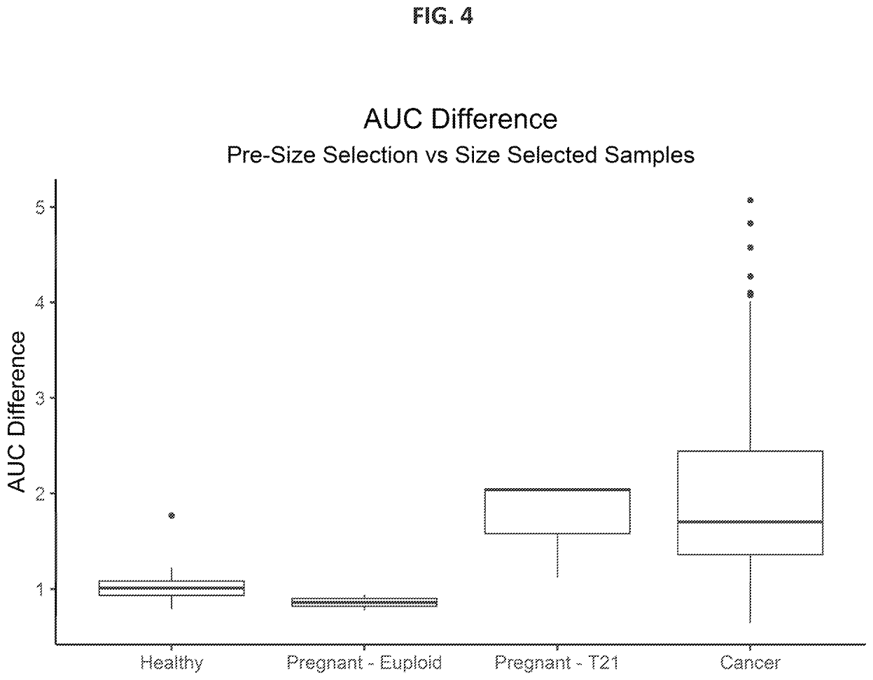

Copy number alterations (CNAs) were identified in the cfDNA data and characterized using analytical methods originally developed for noninvasive prenatal testing and subsequently optimized for ctDNA. The amplitude of a detectable autosomal CNA represents the relative magnitude of the CNA. As is shown in FIG. 4, when evaluating cfDNA from healthy patients, the amplitudes of CNAs before and after size selection were on average within 6%, consistent with a lack of signal enrichment in the absence of disease. Conversely, detectable CNAs in cancer patients were on average 47% greater in amplitude in size selected samples than in the same samples prior to size selection, consistent with an enrichment of signal. These data demonstrate a proof-of-concept for using size selection to enhance signal for the detection of tumor-specific variants in cancer patients.

### Example 3

Cell free DNA was size selected and CNAs were identified as described above, and the average AUC difference for each sample pre- and post-size selection was calculated. The AUC difference was calculated for each copy number alteration in all samples by dividing the AUC of a particular copy number alteration post size selection by the AUC of the same copy number alteration pre size selection. In healthy patients, there were 15 copy number alterations found in both pre and post size selected samples. These were likely germline CNAs and the average AUC difference was 0.95, which is consistent with a lack of signal enrichment in the absence of disease. In cancer patients, there were a total of 172 CNAs found in both pre and post size selected samples and the average AUC difference was 2.03, which is consistent with an enrichment of signal from tumor derived cell free DNA.

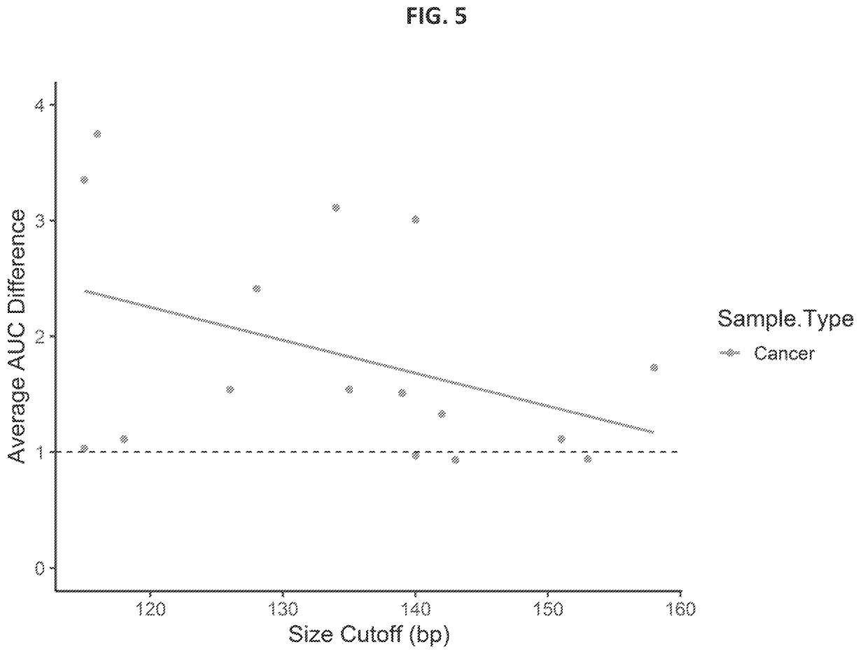

Furthermore, as is shown in FIG. 5, all detected CNAs for a for each of sixteen different cancer patients were analyzed pre- and post-size selection, and the AUC difference for each patient was calculated. Each point in the figure represents the average AUC difference of all detected CNAs for a single patient. As the size cutoff increases, the average AUC difference decreases. If, alternatively, the AUC difference had remained at an average of around 1 despite size selection, there is a high probability the detected CNAs are germline.

### Example 4

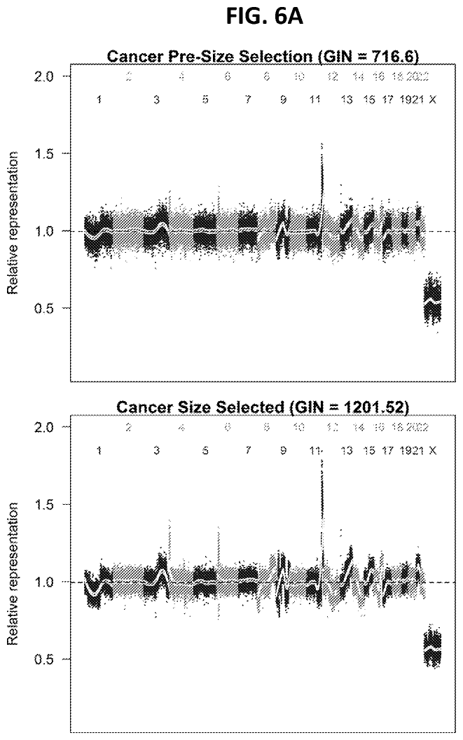

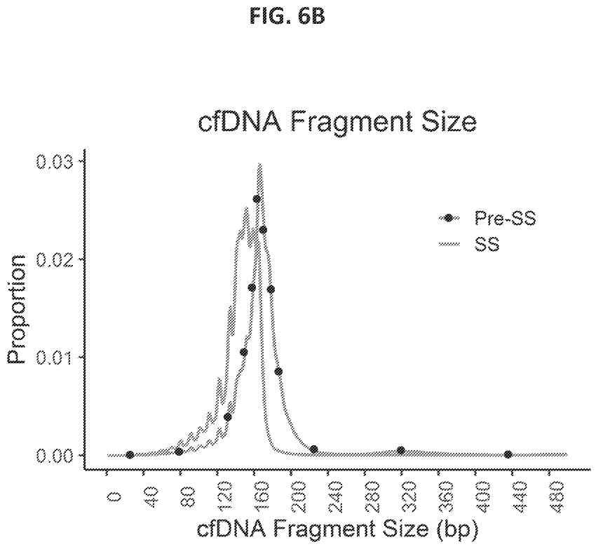

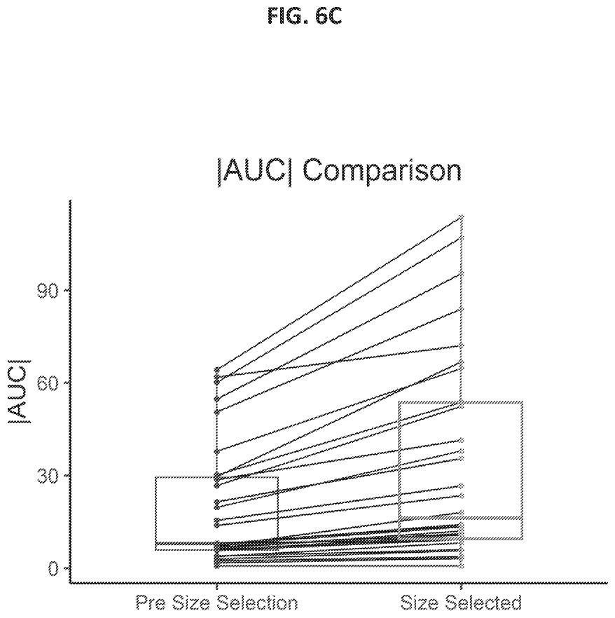

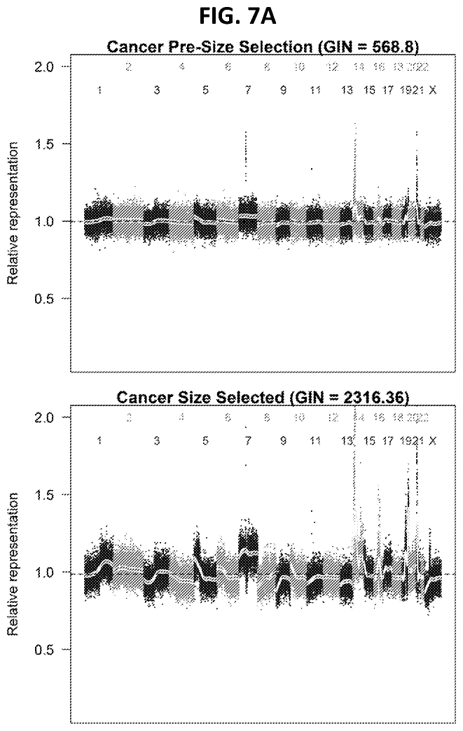

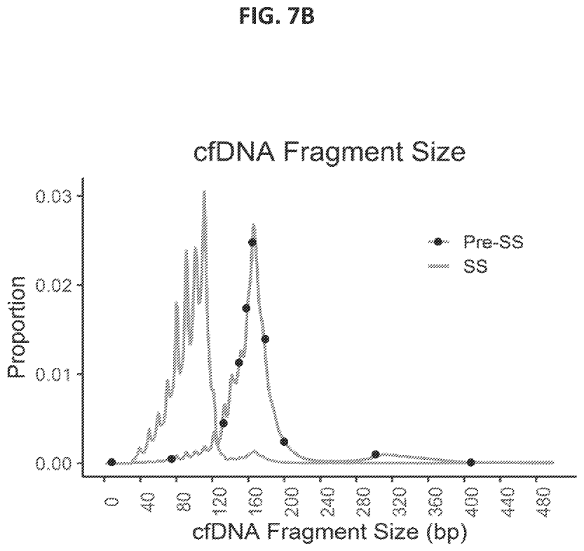

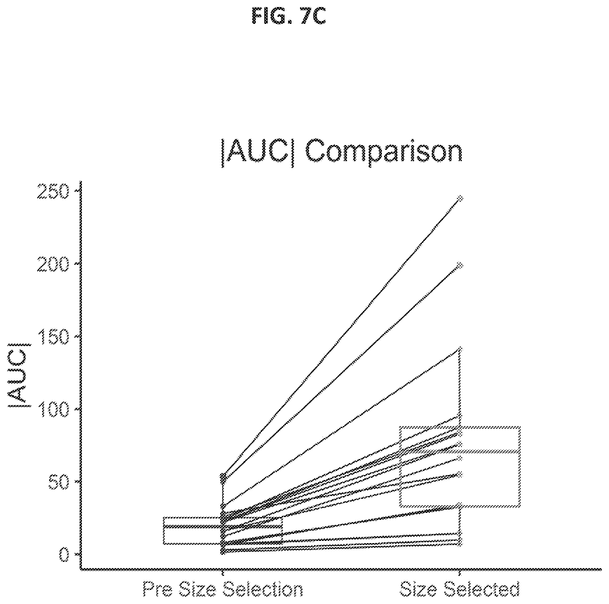

Cell free DNA was size selected and CNAs identified as described above, and the AUC difference pre- and post-size selection was calculated for each CNA. Two different size selection criteria were used: a high size cutoff of 152 bp, depicted in FIGS. 6A, 6B and 6C, and a low size cutoff of 116 bp, depicted in FIGS. 7A, 7B and 7C.

A very high size selection cutoff of 152 bp was used on the sample depicted in FIGS. 6A, 6B and 6C. FIG. 6A shows the genome-wide profiles of the sample before (top panel) and after (lower panel) size selection where CNAs increased in magnitude only slightly and the GIN increased some after size selection. Chromosome numbers corresponding each's respective location in the genome-wide profile are notated across the tops of the upper and lower panels. FIG. 6B shows the cfDNA fragment size profile of the sample before and after size selection and the size selected sample still contains a large portion of the sample before size selection. There were many copy number alterations found both pre- and post-size selection. There was some enrichment of CNAs post-size selection but, on average, AUC was 1.7× greater, compared to the overall average of 2.03× greater. FIG. 6C shows the absolute value of the AUC for each CNA detected pre-size selection on the left and post-size selection on the right.

A low size selection cutoff of 116 bp was used on the sample depicted in FIGS. 6A, 6B and 6C. Using the lower cutoff for size selection, it was found that, there was a large shift in size in the fragment size profile following size-selection. Thus, FIG. 7A shows the genome-wide profiles of the sample before (upper panel) and after (lower panel) size selection where CNAs increased significantly in magnitude and the GIN increased significantly after size selection. As is shown in FIG. 7C, copy number alterations post-size selection were clearly amplified, with an average difference in AUC of 3.7× greater than pre-size selection. Examples of CNAs can be seen on chromosome 7, where the entire chromosome is amplified post-size selection, as is shown in the bottom half of FIG. 7A. Note that the three obvious amplifications on chromosome 7, 14, and 21 pre-size selection, depicted in the top half of FIG. 7A, are so large after size selection that they have gone above the limits of this figure.

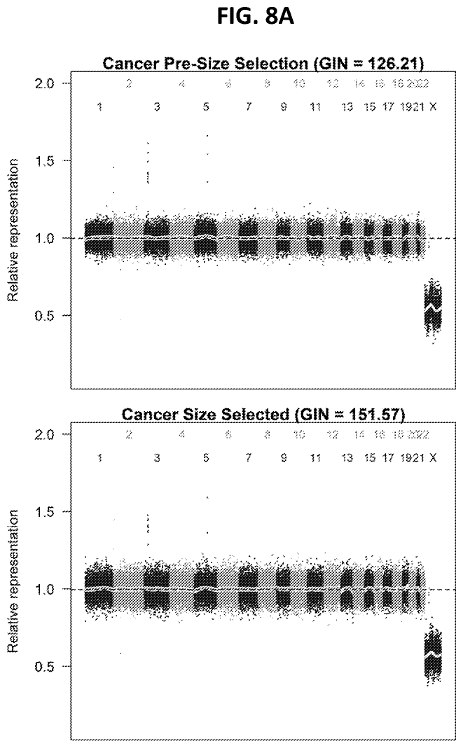

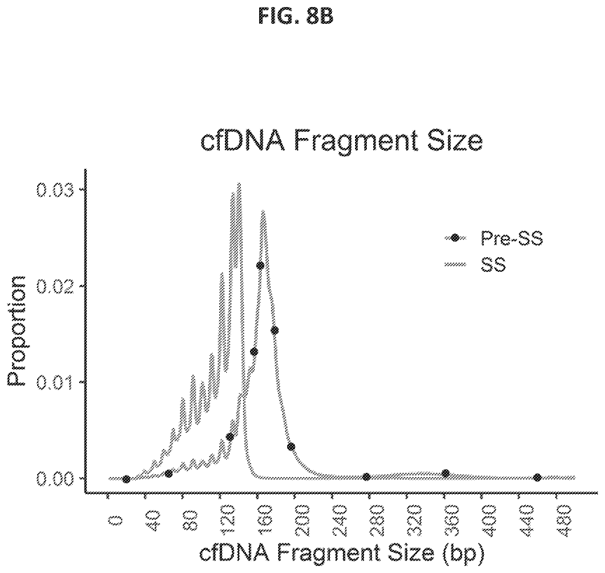

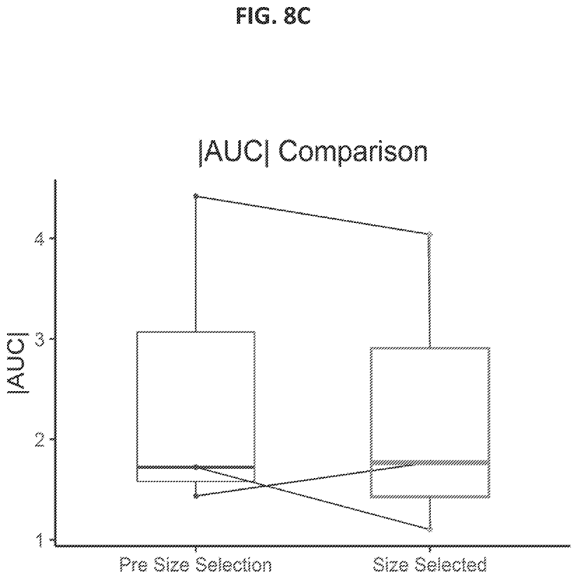

Finally, an example of a sample from a healthy patient is depicted in FIGS. 8A, 8B and 8C. When copy number alterations are likely germline, such as in this healthy patients or in cancer patients with lower tumor burden, the AUC does not change much between pre and post size selection, as can be seen in FIG. 7C. The average AUC change for this sample was 0.93.

### Example 5— Examples of Certain Embodiments

Listed hereafter are non-limiting examples of certain embodiments of the technology.

A1. A method for analyzing circulating cell-free nucleic acids from a subject comprising
(a) obtaining a sample comprising circulating cell-free nucleic acid fragments from the subject; and
(b) preparing a library from the sample, wherein the library comprises the circulating cell-free nucleic acid fragments ligated to at least one adapter.
   A2. The method of embodiment A1, further comprising selecting for adapter-ligated nucleic acids having a subject cell-free nucleic acid fragment that is less than 165 bp, or optionally less than 160 bp, or optionally less than 155 bp, or optionally less than 150 bp, or optionally less than 145 bp.
   A3. The method of embodiment A2, further comprising selecting for adapter-ligated nucleic acids having a subject cell-free nucleic acid fragment that is greater than 15 bp.
   A4. The method of embodiment A3, further comprising determining the sequence of the selected subject nucleic acid fragments.
   A5. The method of embodiment A4, further comprising quantifying copy number alternations (CNAs) in the sequenced subject nucleic acid fragments.
   A6. The method of any of the preceding embodiments, wherein the sample is a plasma sample.
   A7. The method of any of the preceding embodiments, wherein the circulating cell-free nucleic acid fragments comprise circulating tumor DNA (ctDNA).
   A8. The method of any of embodiments A1 to A6, wherein the circulating cell-free nucleic acid fragments comprise circulating fetal cell-free DNA (fetal cfDNA).
   A9. The method of any of embodiments A1 to A6, further comprising determining a status of the subject based on the selected subject nucleic acid fragments.
   A10 The method of any of the preceding embodiments, further comprising determining a status of the subject based on the CNAs present in the selected subject nucleic acid fragments
   A11. The method of any of the preceding embodiments, wherein the level of CNAs are quantified using a genomic instability number (GIN).
   A12. The method of embodiment A9 to A11, wherein the status of the subject is a presence or absence of a cancer.
   A13. The method of embodiment A9 to A11, wherein the status of the subject is a progression of a cancer.
   A14. The method of embodiment A9 to A11, wherein the status of the subject is a remission of a cancer.
   A15. The method embodiment A9 to A11, wherein the status of the subject is pregnant with a fetus exhibiting an aneuploidy.
   A16. The method of any of the preceding embodiments, wherein the adapter-ligated nucleic acid fragments are size selected via electrophoresis.
   A17. The method of any of embodiments A1 to A15, wherein the adapter-ligated nucleic acid fragments are size selected via magnetic bead-based selection
   A18. The method of any of embodiments A1 to A15, wherein the adapter-ligated nucleic acid fragments are size selected in silico during the processing of sequencing data.
   A19. The method of any of embodiments A2 to A18, wherein the subject cell free nucleic acid fragments are less than 143 bp.
   A20. The method of any of the preceding embodiments, wherein the method comprises the analysis of multiplexed samples.
   B1. A system for analyzing circulating cell free nucleic acids by any of the methods of any of the embodiments A1 to A20.
   C1. A computer-program product for analyzing circulating cell free nucleic acids by any of the methods of any of the embodiments A1 to A20, or the system of embodiment B1.
   D1. A composition for analyzing circulating cell-free nucleic acids from a subject comprising a library of circulating cell-free nucleic acids ligated to at least one adaptor.
   D2. The composition of embodiment D1, wherein the library comprises adapter-ligated cell-free nucleic acid fragments that are less than 165 bp, or optionally less than 160 bp, or optionally less than 155 bp, or optionally less than 150 bp, or optionally less than 145 bp.
   D3. The composition of any of embodiments D1 or D2, wherein the library comprises adapter-ligated nucleic acids having a subject cell-free nucleic acid fragment that is greater than 15 bp.

That which is claimed is:

1. A method for analyzing circulating cell-free nucleic acids from a subject comprising
(a) obtaining a sample comprising circulating cell-free nucleic acid fragments from the subject; and
(b) preparing a library from the sample, wherein the library comprises the circulating cell-free nucleic acid fragments ligated to at least one adapter.

2. The method of claim 1, further comprising selecting for adapter-ligated nucleic acids having a subject cell-free nucleic acid fragment that is less than 150 bp.

3. The method of claim 2, further comprising selecting for adapter-ligated nucleic acids having a subject cell-free nucleic acid fragment that is greater than 15 bp.

4. The method of claim 3, further comprising determining the sequence of the selected subject nucleic acid fragments.

5. The method of claim 4, further comprising quantifying copy number alternations (CNAs) in the sequenced subject nucleic acid fragments.

6. The method of claim 1, wherein the sample is a plasma sample.

7. The method of claim 1, wherein the circulating cell-free nucleic acid fragments comprise circulating tumor DNA (ctDNA).

8. The method of claim 1, wherein the circulating cell-free nucleic acid fragments comprise circulating fetal cell free DNA (fetal cfDNA).

9. The method of claim 3, further comprising determining a status of the subject based on the selected subject nucleic acid fragments.

10. The method of claim 5, further comprising determining a status of the subject based on CNAs present in the selected subject nucleic acid fragments.

11. The method of claim 10, wherein the status of the subject is a presence or absence of a cancer.

12. The method of claim 10, wherein the status of the subject is a progression of a cancer.

13. The method of claim 10, wherein the status of the subject is a remission of a cancer.

14. The method claim 10, wherein the status of the subject is pregnant with a fetus exhibiting an aneuploidy.

15. The method of claim 5, wherein the level of CNAs are quantified using a genomic instability number (GIN).

16. The method of claim 2, wherein the adapter-ligated nucleic acid fragments are size selected via at least one of electrophoresis, magnetic bead-based selection, or in silico during the processing of sequencing data.

17-18. (canceled)

17. The method of claim 2, wherein the subject cell free nucleic acid fragments are less than 143 bp.

18. The method of claim 1, wherein the library comprises multiplexed samples.

19. A system for analyzing circulating cell free nucleic acids from a subject by:
a station for obtaining a sample comprising circulating cell-free nucleic acid fragments from the subject and preparing a library from the sample, wherein the library comprises the circulating cell-free nucleic acid fragments ligated to at least one adapter; and
a station for selecting for adapter-ligated nucleic acids having a subject cell-free nucleic acid fragment that is less than 150 bp.

22. (canceled)

20. A composition for analyzing circulating cell-free nucleic acids from a subject comprising a library of circulating cell-free nucleic acids ligated to at least one adaptor, wherein the adapter-ligated nucleic acids are less than 150 bp in size.
